(19) United States
(12) Patent Application Publication
(10) Pub. No.: US 2005/0021090 A1
(43) Pub. Date: Jan. 27, 2005

(54) Method and device for regulation of limbic system of the brain by means of neuro-electrical coded signals

(76) Inventors: Eleanor Schuler, Rio Ranche, NM (US); Claude K. Lee, Reno, NV (US)

Correspondence Address: JENNIFER L. BALES, MOUNTAIN VIEW PLAZA, 1520 EUCLID CIRCLE, LAFAYETTE, CO, 80026-1250, US

(21) Appl. No.: 10/897,700

(22) Filed: Jul. 23, 2004

Related U.S. Application Data
(60) Provisional application No. 60/489,839, filed on Jul. 23, 2003.

(51) Int. Cl. A61N 1/18

(52) U.S. Cl. 607/2

(57) ABSTRACT

A method and device for limbic system control to provide medical treatment for emotions and instinct. The method comprises selecting neuro-electrical coded signals from a storage area that are representative of limbic brain structure function. The selected neuro-electrical coded signals are then transmitted to a treatment member, which is in direct contact with the body, and which then broadcasts the neuro-electrical coded signals to a specific nerve or organ to modulate the limbic system functioning. A control module is provided for transmission to the treatment member. The control module contains the neuro-electrical coded signals which are selected and transmitted to the treatment member, and computer storage can be provided for greater storage capacity and manipulation of the neuro-electrical coded signals.

### RELATED APPLICATIONS

This is the non-provisional filing of provisional application Ser. No. 60/489,839, filed Jul. 23, 2003, entitled “Method for Regulation of Limbic System of the Brain by Means of Neuro-Coded Signals.”

### BACKGROUND OF THE INVENTION

This invention relates to a device and method for regulation of the limbic system of the brain by means of neuro-electrical coded signals.

The emotional and instinctual system of the human or animal brain has been designated as the limbic system. It controls inborn and acquired behavior. It is the seat of emotions and motivation. Such system consists of a grouping of structures described as the limbic cortex which is located near the brainstem, more-or-less in the center of the brain. The Latin word “limbus” denotes “a border” which describes the ring of gyri that surrounds the brainstem as the locus of the limbic anatomical structures. The thinking part of the brain, called the cerebral cortex, which surrounds the structures identified as being part of the limbic system are able to modulate to a certain degree the instinctual & emotional outbursts or impulsive activity of the limbic system. Most mental illnesses have there basis in malfunction of the limbic system. Historically the limbic system was known as the reptilian brain.

The structures comprising the Limbic system are linked at a minimum by neuronal circuits to include, at a minimum, the hypothalamus; hippocampus and the parahippocampal gyrus; mamillary bodies; anterior & medial thalamic nuclei; cingulate gyrus; septal pellucidum area; nucleus accumbens; olfactory tract & bulb; neocortical area which include the amygdaloid body and the orbito-frontal cortex. There may be other close located structures which participate, such as the pons and medulla oblongata, which initiate changes in the vital organs to match the emotional and instinctual impulses operating in the limbic system during a period of time. Such changes, controlled by the limbic system, include increase in heart rate & blood pressure, as well as cessation of digestive activity during flight or fight impulses.

The senses of smell, vision and hearing converge their information into the neuronal processes of the limbic system, along with positional and motor activity status information.

The limbic instinctual & emotional activity includes thirst, hunger (food & water intake desire &limits); concept of body temperature (hot or cold); blushing or skin paleness; urge to defecate or urinate; anger or rage; mamillary-gland milk output and licking of lips and swallowing. In addition, anxiety & fear; tameness & placid feelings and learning stimulations along with other behavior activity emanate from the limbic system. Eating, chewing, licking and swallowing of food and the drinking of water and other drinks are impulses that are directed by the limbic system. Sexual & reproductive drive and copulatory strategy and function such as control of ejaculation of the male and ovulation & uterine contractions in the female are controlled largely by the limbic system.

Since most of the emotional & instinctual disorders along with most of the mental disorders seem to originate from malfunction of the limbic system, the ability to control the neuronal impulses connected with such medical and psychiatric conditions may offer a cure or result in some better control of such disorders. Presently the state-of-the-art for medically treating malfunction and disease of the limbic anatomical region is by medication and psychiatric support. In addition, very limited surgical intervention, electric shock or radiation therapy are available treatment modalities.

The ability to electrically adjust or regulate the limbic activities to cease or even to partially modulate undesirable emotional and instinctual urges would be a compelling medical technology for potentially controlling or adjusting out-of -control or socially unacceptable urges. The control of the neuro-circuits of the limbic system can be done by means of neuro-electrical coded signals (waveforms) that would replace aberrant neuro-electrical coded signals with the normally expected signal as a means of medical treatment. The ability to influence stress response, sexual function, and other behavior(s) by changing neuro-electrical coded signals that influence limbic system output urges, would help treat many mental illnesses. Influence of endocrine glands from limbic signals could also help better balance the endocrine secretions which are thought to be the cause of many mental and sexual disturbances. Such control of emotion and aberrant instinctual behavior by actual neuro-electrical signals generated by a device that records, stores and rebroadcast them would greatly add to the clinical medicine tools of the psychiatrist. Such limbic control technology would provide a clinical neuro-electric method to fine-tune the function of many so-called mental conditions that would be for the greater benefit of mankind. It also can be used to assist in rescuing emergency room patients by bringing emotional dysfunction of rage, for example, to a more placid status that may help the patient towards recovery. The invention could be part of a nervous system-wide treatment computerized device that correct malfunctions of emotion or instinctual events of a psychiatric nature.

The treatment method and device would use the actual neuro-electrical coded signals that send operational information to operate and regulate the wide variety of limbic system needs and impulses of the human and animal body. These actual neuron signals travel along selected nerves to send the operational commands to the target structure within the limbic system and as transmitted outwardly to other organs, muscles and glands which carry out the instructions from the limbic system.

The glands activated by the limbic system of the human and other mammals are operated by neuro-electric signals from the brain which, in turn can turn-on, in selected cases, chemical instructional signals. When such signals from the limbic system turn-on chemical signals which travel to target organs via the blood stream, they cause the will of the emotional or instinctual impulses of the limbic output to be carried out.

### SUMMARY OF THE INVENTION

The invention provides a method for controlling the limbic system. Stored neuro-electrical coded signals that are generated and carried in the body are selected from a storage area. The selected waveforms are then transmitted to a treatment member which is in direct contact with the body. The treatment member then broadcasts the selected neuro-electrical coded signals to an area in the body.

The neuro-electrical coded signals may be selected from a storage area in a computer, such as a scientific computer. The process of transmitting the selected neuro-electrical coded signals can either be done remotely or with the treatment member connected to a control module. The transmission may be seismic, electronic, or via any other suitable method.

The invention further provides an apparatus for controlling the limbic system. The apparatus includes a source of collected neuro-electrical coded signals that are indicative of limbic functioning, a treatment member in direct contact with the body, means for transmitting collected waveforms to the treatment member, and means for broadcasting the collected neuro-electrical coded signals from the treatment member to an area in the body.

The transmitting means may include a digital to analog converter. The source of collected waveforms preferably comprises a computer which has the collected waveforms stored in digital format. The computer may include separate storage areas for collected neuro-electrical coded signals of different categories.

The treatment member may be comprised of an antenna or an electrode, or any other means of broadcasting one or more neuro-electrical coded signals directly to the body.

### BRIEF DESCRIPTION OF THE DRAWINGS

The invention is described in greater detail in the following description of examples embodying the best mode of the invention, taken in conjunction with the drawing figures, in which:

FIG. 1 is a schematic diagram of one form of apparatus for practicing the method according to the invention;

FIG. 2 is a schematic diagram of another form of apparatus for practicing the method according to the invention; and

FIG. 3 is a flow chart of the method according to the invention.

### DESCRIPTION OF EXAMPLES EMBODYING THE BEST MODE OF THE INVENTION

For the purpose of promoting an understanding of the principles of the invention, references will be made to the embodiments illustrated in the drawings. It will, nevertheless, be understood that no limitation of the scope of the invention is thereby intended, such alterations and further modifications in the illustrated device, and such further applications of the principles of the invention illustrated herein being contemplated as would normally occur to the one skilled in the art to which the invention relates.

Skin usually has a 1000 to 30,000 ohm resistance while the interior of the body is quite conductive. All coded signals operate at less than 1 volt, naturally. Applied voltage may be up to 20 volts according to the invention to allow for voltage loss during the transmission or conduction of the required coded signals. Current should always be less than 2 amps output for the invention. Direct conduction into the nerves via electrodes connected directly to such nerves will likely have outputs of less than 3 volts and current of less than one-tenth of an amp. Up to 10 or more channels may be used simultaneously to exert medical treatment on limbic nerve control to aid a patient in moving or performing tasks suitable to his or her well-being as medical treatment.

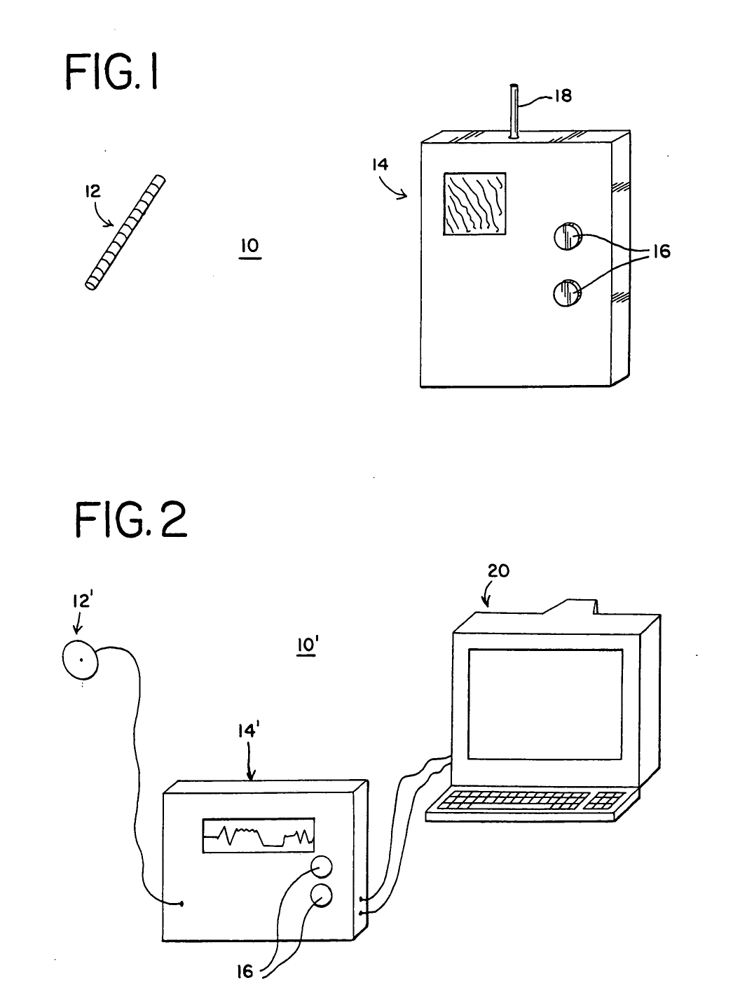

The invention encompasses both a device and a method for limbic system control by means of neuro-electrical coded signals waveforms. One form of a device 10 for limbic system control, as shown in FIG. 1, is comprised of at least one treatment member 12, and a control module 14. The treatment member 12 is in direct contact with a body and receives a neuro-electrical coded signal from the control module 14. The treatment member 12 may be an electrode, antenna, a seismic transducer, or any other suitable form of conduction attachment for broadcasting limbic nerve signals that regulate or operate limbic function in human or animals. The treatment member 12 may be attached to efferent nerves leading to the limbic nerve system, afferent nerves leading to the brain to accomplish modulation of limbic output, the cervical spine, the neck, or any limbic nerve in a surgical process. Such surgery may be accomplished with “key-hole” entrance in a thoriac or limb stereo-scope procedure. If necessary a more expansive thoracotomy approach may be required for more proper placement of the treatment member 12. Neuro-electrical coded signals known to limbic system function may then be sent into nerves that are in close proximity with the brain stem.

The control module 14 is comprised of at least one control 16, and an antenna 18. The control 16 allows the device to regulate the signal transmission into the body. As shown in FIG. 1, the control module 14 and treatment member 12 can be entirely separate elements allowing the device 10 to be operated remotely. The control module 14 can be unique, or can be any conventional device which can provide neuro-electrical coded signals for transmission to the treatment member 12.

In an alternate embodiment of the device 10, as shown in FIG. 2, the control module 14′ and treatment member 12′ are connected. Similar members retain the same reference numerals in this figure. Additionally, FIG. 2 further shows another embodiment of the device 10′ as being connected to a computer 20, which provides greater capacity to store the neuro-electrical coded signals. The output voltage and amperage provided by the device 10′ during treatment shall not exceed 20 volts nor 2 amps for each signal.

The computer 20 is used to store the unique neuro-electrical coded signals, which are complex and unique to each the limbic system and function of the limbic system. It is a neuro-electrical coded signal(s) selected from the stored library of waveforms in the computer 20 which is transmitted to the control module 14′ and used for treatment of a patient. The waveform signals, and their creation, are described in greater detail in U.S. patent application Ser. No. 10/000,005, filed Nov. 20, 2001, and entitled “Device and Method to Record, Store, and Broadcast Specific Brain Waveforms to Modulate Body Organ Functioning,” the disclosure of which is incorporated herein by reference.

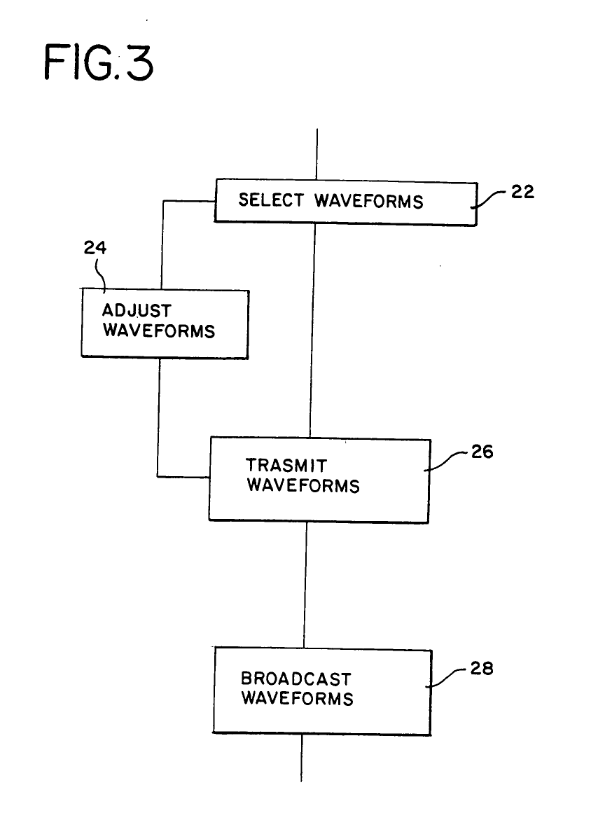

The invention further includes a method, as shown in FIG. 3, for using the device 10, 10′ for limbic system control. The method begins at step 22 by selecting one or more stored neuro-electrical coded signals from a menu of cataloged neuro-electrical coded signals. The neuro-electrical coded signals selected activate, deactivate, or adjust the muscular system. Such neuro-electrical coded signals are similar to those naturally produced by the brain structures for balancing and controlling limbic processes. Once selected, the neuro-electrical coded signals may be adjusted, in step 24, to perform a particular function in the body. Alternatively, if it is decided that the neuro-electrical coded signals do not need to be adjusted, step 24 is skipped and the process proceeds directly with step 26. At step 26, the neuro-electrical coded signal is transmitted to the treatment member 12, 12′ of the device 10, 10′.

Upon receipt of the neuro-electrical coded signals, the treatment member 12, 12′ broadcasts the neuro-electrical coded signals to the appropriate limbic nerve or nerve location, as shown in step 28. The device 10, 10′ utilizes appropriate neuro-electrical coded signals to adjust or modulate limbic action via conduction or broadcast of electrical signals into selected nerves. Controlling limbic system function may require sending neuron electrical coded signals into one or more nerves, including up to ten nerves simultaneously. It is believed that target nerves “respond” to their own individual neuro-electrical coded signals.

In one embodiment of the invention, the process of broadcasting by the treatment member 12, 12′ is accomplished by direct conduction or transmission through unbroken skin in a selected appropriate zone on the neck, head, limb(s), spine, or thorax. Such zone will approximate a position close to the nerve or nerve plexus onto which the signal is to be imposed. The treatment member 12, 12′ is brought into contact with the skin in a selected target area that allows for the transport of the signal to the target nerve(s).

In an alternate embodiment of the invention, the process of broadcasting the neuro-electrical coded signal is accomplished by direct conduction via attachment of an electrode to the receiving nerve or nerve plexus. This requires a surgical intervention as required to physically attach the electrode to the selected target nerve. Direct implantation on the nervous system of the selected nerve or nerve ganglion may be performed in order to transmit signals to control all or some nerve function. Such implantation can be presynaptic or post synaptic and may be attached to ganglion or nerve plexis associated with the desired limbic function.

In yet another embodiment of the invention, the process of broadcasting is accomplished by transposing the neuro-electrical coded signal into a seismic form where it is sent into a region of the head, neck, limb(s), spine, or thorax in a manner that allows the appropriate “nerve” to receive and to obey the coded instructions of such seismic signal. The treatment member 12, 12′ is pressed against the unbroken skin surface using an electrode conductive gel or paste medium to aid conductivity.

Various features of the invention have been particularly shown and described in connection with the illustrated embodiments of the invention. However, it must be understood that these particular products, and their method of manufacture, do not limit but merely illustrate, and that the invention is to be given its fullest interpretation within the terms of the appended claims.

1. A method for controlling the limbic system comprising the steps of:
a. selecting from a storage area one or more waveforms generated in the body and carried by neurons in the body;
b. transmitting or conducting the selected waveforms to a treatment member in contact with the body; and
c. broadcasting the selected waveforms from the treatment member to an area in the body that is affected to control the limbic system.

2. The method according to claim 1, in which step “a” further includes selecting said waveforms from a storage area in a computer.

3. The method according to claim 1, in which step “b” further comprises transmitting the selected waveforms remotely to the treatment member.

4. The method according to claim 1, in which step “b” further comprises seismic transmission of the selected waveforms.

5. An apparatus for controlling the limbic system, comprising:
a. a source of collected waveforms generated in the body and indicative of limbic functioning;
b. a treatment member adapted to be in direct contact with the body;
c. means for transmitting one or more of the collected waveforms to the treatment member; and
d. means for broadcasting the collected waveforms from the treatment member to an area in the body such that a nerve is affected, thereby controlling the limbic system.

6. The apparatus according to claim 5, in which said transmitting means includes a digital to analog converter.

7. The apparatus according to claim 5, in which said source comprises a computer having collected waveforms stored in digital format.

8. The apparatus according to claim 7, in which said computer includes separate storage areas for collecting waveforms of different limbic nerve functional categories.

9. The apparatus according to claim 5, in which the treatment member comprises an antenna for broadcasting limbic nerve signals.

10. The apparatus according to claim 5, in which the treatment member comprises an electrode.
